(19) United States
(12) Patent Application Publication
(10) Pub. No.: US 2018/0104507 A1
(43) Pub. Date: Apr. 19, 2018

(54) OPTOGENETIC THERAPEUTIC AND METHOD OF TREATING RETINAL DEGENERATIVE AND NEURODEGENERATIVE DISEASES

(71) Applicant: Sheila Nirenberg, New York, NY (US)

(72) Inventor: Sheila Nirenberg, New York, NY (US)

(21) Appl. No.: 15/786,886

(22) Filed: Oct. 18, 2017

Related U.S. Application Data
(60) Provisional application No. 62/410,200, filed on Oct. 19, 2016.
(60) Provisional application No. 62/410,519, filed on Oct. 20, 2016.

(51) Int. Cl. A61N 5/06; G01N 33/50; C07K 14/47; A61K 41/00

(52) U.S. Cl. CPC A61N 5/062; G01N 33/5058; A61F 9/00; A61K 41/0057; C07K 2319/60; C07K 14/4702

(57) ABSTRACT

A method of treating a subject afflicted with a retinal degenerative or neurodegenerative disease including administering an optogenetic therapeutic to the subject, where the optogenetic therapeutic includes an optogenetic protein fused or linked to a marker protein. An optogenetic therapeutic for use in treating a subject afflicted with a retinal degenerative or neurodegenerative disease, including an optogenetic protein fused or linked to a marker protein.

### CROSS-REFERENCE TO RELATED APPLICATIONS

The present application claims priority to U.S. Provisional Patent Application Nos. 62/410,200, filed Oct. 19, 2016, and 62/410,519, filed Oct. 20, 2016, the disclosures and teachings of which are incorporated herein by reference.

### TECHNICAL FIELD

The present invention relates to an optogenetic therapeutic. In particular, the present invention relates to a fused or linked optogenetic protein-marker protein product that serves as an optogenetic therapeutic in treating retinal degenerative or neurodegenerative diseases.

### BACKGROUND OF THE INVENTION

Optogenetics represents a powerful approach for controlling neural activity, with numerous applications in both basic and clinical science. With respect to the basic science, it provides a method for teasing apart circuit operations, as it allows circuit components to be turned on or off with very high spatial, e.g., single cell level, and temporal, e.g., millisecond, resolution. Regarding clinical applications, it presents a potential avenue for new types of selective treatments, specifically mechanisms that activate or inactivate specific components in a damaged or malfunctioning circuit and re-engage them into normal activity.

Much of the nonclinical testing of optogenetic proteins, such as a channelrhodopsin (ChR), utilize optogenetic proteins that are fused or linked to marker proteins, such as green fluorescent protein (GFP). Linking an optogenetic protein like ChR to a marker protein makes it easier for an investigator to identify ChR-expressing cells for stimulation and electrophysiological recording. For example, if one wants to test the effectiveness of ChR in a tissue containing millions of cells, one approach might be to infect the tissue with a viral vector expressing the ChR gene as well as GFP or use a ChR-GFP fusion gene. The marker gene would permit the investigator to find the ChR-expressing cells, as ChR itself is not fluorescent or readily detectable. However, a therapeutic treatment utilizing a ChR-GFP fusion protein rather than ChR alone is discouraged with the predominant expectation that regulatory agencies would not permit usage of GFP or similar marker protein in patients, as additional risk relating to usage of a marker gene unless it offers therapeutic benefit. If, however, the marker gene also offers therapeutic value, such as by (a) facilitating the delivery of the ChR-marker fusion protein to the cell membrane, which increases its effectiveness, (b) increasing current into the cell, which also increases effectiveness of the therapeutic, or (c) increasing stability of the therapeutic over time, then usage of the ChR-marker fusion protein may be superior, as it both increases the effectiveness of the therapeutic and reduces potential risk for the patient because the therapeutic may be used at lower doses and still achieve the same level of activity. The field of ChR proteins is reviewed generally in Gradinaru et al., “Molecular and Cellular Approaches for Diversifying and Extending Optogenetics,” Cell (2010).

### SUMMARY OF THE INVENTION

In general, in one aspect, the invention features a method of treating a subject afflicted with a retinal degenerative or neurodegenerative disease including administering an optogenetic therapeutic to the subject, where the optogenetic therapeutic includes an optogenetic protein fused or linked to a marker protein.

Implementations of the invention may include one or more of the following features. The optogenetic protein may be a channelrhodopsin (ChR). The marker protein may be green fluorescent protein (GFP), enhanced green fluorescent protein (EGFP), yellow fluorescent protein (YFP), or a green fluorescent protein (GFP) derivative. The retinal degenerative or neurodegenerative disease may be retinitis pigmentosa or macular degeneration. The optogenetic therapeutic may be administered to retinal output cells of the subject. The optogenetic therapeutic may be administered via injection. The method may further include administering a device to the subject, where the device is configured to take in images via a camera, compress and encode the images, and transmit coded signals to retinal output cells of the subject. The device may be embedded in or on a pair of eyeglasses that is worn by the subject.

In general, in another aspect, the invention features an optogenetic therapeutic for use in treating a subject afflicted with a retinal degenerative or neurodegenerative disease, including an optogenetic protein fused or linked to a marker protein.

Implementations of the invention may include one or more of the following features. The optogenetic protein may be a channelrhodopsin (ChR). The marker protein may be green fluorescent protein (GFP), enhanced green fluorescent protein (EGFP), yellow fluorescent protein (YFP), or a green fluorescent protein (GFP) derivative. The optogenetic therapeutic may be configured for administration to retinal output cells of the subject. The optogenetic therapeutic may be configured for administration via injection.

In general, in another aspect, the invention features a method of treating a subject afflicted with a retinal degenerative or neurodegenerative disease including administering an optogenetic therapeutic to the subject, where the optogenetic therapeutic comprises Chronos (ChR90) fused or linked to a fluorescent marker protein.

Implementations of the invention may include one or more of the following features. The fluorescent marker protein may be green fluorescent protein (GFP), enhanced green fluorescent protein (EGFP), yellow fluorescent protein (YFP), or a green fluorescent protein (GFP) derivative. The retinal degenerative or neurodegenerative disease may be retinitis pigmentosa or macular degeneration. The optogenetic therapeutic may be administered to retinal output cells of the subject. The optogenetic therapeutic may be administered via injection. The method may further include administering a device to the subject, where the device is configured to take in images via a camera, compress and encode the images, and transmit coded signals to retinal output cells of the subject. The device may be embedded in or on a pair of eyeglasses that is worn by the subject.

### DETAILED DESCRIPTION OF THE INVENTION

This invention provides a method of treating a patient afflicted with a retinal degenerative or neurodegenerative disease including administering an optogenetic therapeutic to the patient, where the optogenetic therapeutic includes an optogenetic protein fused or linked to a marker protein.

It has been presently determined that when a marker protein, e.g., GFP, is fused to an optogenetic protein, e.g., ChR, rather than simply being co-expressed, the marker protein confers additional benefits and advantages on the optogenetic protein. For example, when fused with GFP, ChR appears to be more stabilized in the cells, is trafficked to the targeted location of the membrane, and does not form aggregates when packaged into viral vectors, such as adeno-associated viral vectors. In light of these benefits and advantages, the fusion protein of ChR and GFP, or a GFP derivative, acts as a superior therapeutic agent compared to ChR alone. This superior nature of the ChR-GFP fusion protein for therapeutic purposes has not previously been shown in the clinical field.

Similarly, by fusing GFP with ChR, vectors with ChR transgenes may be made at higher titers with less aggregation. Additionally, the activity of ChR may be more effective as a result of better trafficking to the membrane and better stability upon arrival. Therefore, fusing GFP or its variants with ChR has significant therapeutic merit that is independent of GFP simply serving as a marker.

In a preferred embodiment, the ChR is Chronos, or ChR90. Additionally, while the ChR-GFP fusion has been discussed above, similar fusions of varied optogenetic proteins and marker proteins, including fluorescent marker proteins such as EGFP or YFP fused with ChR, may also be utilized as therapeutic agents in treating retinitis pigmentosa, macular degeneration, and other retinal degenerative or neurodegenerative diseases.

In one embodiment of the present invention, the optogenetic therapeutic is configured for administration, and subsequently administered, to retinal output cells of a patient. Additionally, this administration may be performed via an injection to the retinal output cells that delivers the optogenetic therapeutic.

Finally, the aforementioned therapeutic treatment utilizing the optogenetic therapeutic may be executed in conjunction with administration of a particular device to the patient, with the device being configured to take in images via a camera, compress and encode the images, and transmit coded signals to retinal output cells of the patient. In one embodiment, the device is embedded in or on a pair of eyeglasses that is worn by the patient.

It will be understood by those of ordinary skill in the art that various changes may be made and equivalents may be substituted for elements without departing from the scope of the invention. In addition, many modifications may be made to adapt a particular feature or material to the teachings of the invention without departing from the scope thereof. Therefore, it is intended that the invention not be limited to the particular embodiments disclosed, but that the invention will include all embodiments falling within the scope of the claims.

What is claimed is:

1. A method of treating a subject afflicted with a retinal degenerative or neurodegenerative disease comprising administering an optogenetic therapeutic to the subject, wherein the optogenetic therapeutic comprises an optogenetic protein fused or linked to a marker protein.

2. The method of claim 1, wherein the optogenetic protein is a channelrhodopsin (ChR).

3. The method of claim 1, wherein the marker protein is green fluorescent protein (GFP), enhanced green fluorescent protein (EGFP), yellow fluorescent protein (YFP), or a green fluorescent protein (GFP) derivative.

4. The method of claim 1, wherein the retinal degenerative or neurodegenerative disease is retinitis pigmentosa or macular degeneration.

5. The method of claim 1, wherein the optogenetic therapeutic is administered to retinal output cells of the subject.

6. The method of claim 5, wherein the optogenetic therapeutic is administered via injection.

7. The method of claim 1, further comprising administering a device to the subject, wherein the device is configured to take in images via a camera, compress and encode the images, and transmit coded signals to retinal output cells of the subject.

8. The method of claim 7, wherein the device is embedded in or on a pair of eyeglasses that is worn by the subject.

9. An optogenetic therapeutic for use in treating a subject afflicted with a retinal degenerative or neurodegenerative disease, comprising an optogenetic protein fused or linked to a marker protein.

10. The optogenetic therapeutic of claim 9, wherein the optogenetic protein is a channelrhodopsin (ChR).

11. The optogenetic therapeutic of claim 9, wherein the marker protein is green fluorescent protein (GFP), enhanced green fluorescent protein (EGFP), yellow fluorescent protein (YFP), or a green fluorescent protein (GFP) derivative.

12. The optogenetic therapeutic of claim 9, wherein the optogenetic therapeutic is configured for administration to retinal output cells of the subject.

13. The optogenetic therapeutic of claim 12, wherein the optogenetic therapeutic is configured for administration via injection.

14. A method of treating a subject afflicted with a retinal degenerative or neurodegenerative disease comprising administering an optogenetic therapeutic to the subject, wherein the optogenetic therapeutic comprises Chronos (ChR90) fused or linked to a fluorescent marker protein.

15. The method of claim 14, wherein the fluorescent marker protein is green fluorescent protein (GFP), enhanced green fluorescent protein (EGFP), yellow fluorescent protein (YFP), or a green fluorescent protein (GFP) derivative.

16. The method of claim 14, wherein the retinal degenerative or neurodegenerative disease is retinitis pigmentosa or macular degeneration.

17. The method of claim 14, wherein the optogenetic therapeutic is administered to retinal output cells of the subject.

18. The method of claim 17, wherein the optogenetic therapeutic is administered via injection.

19. The method of claim 14, further comprising administering a device to the subject, wherein the device is configured to take in images via a camera, compress and encode the images, and transmit coded signals to retinal output cells of the subject.

20. The method of claim 19, wherein the device is embedded in or on a pair of eyeglasses that is worn by the subject.
